(19) United States
(12) Patent Application Publication
(10) Pub. No.: US 2014/0033342 A1
(43) Pub. Date: Jan. 30, 2014

(54) ODD-PLOIDY, SEED-PROPAGATED MISCANTHUS x GIGANTEUS

(71) Applicants: Neal I. Gutterson, Oakland, CA (US); Jeffrey P. Klingenberg, Tifton, GA (US); Michael A. Pereira, Morgan Hill, CA (US); Dean E. Engler, Chico, CA (US); Katrin Jakob, Alameda, CA (US)

(72) Inventors: Neal I. Gutterson, Oakland, CA (US); Jeffrey P. Klingenberg, Tifton, GA (US); Michael A. Pereira, Morgan Hill, CA (US); Dean E. Engler, Chico, CA (US); Katrin Jakob, Alameda, CA (US)

(73) Assignee: MENDEL BIOTECHNOLOGY, INC., Hayward, CA (US)

(21) Appl. No.: 14/111,718

(22) PCT Filed: Apr. 13, 2012
(86) PCT No.: PCT/US12/33597

§ 371 Date: Oct. 14, 2013

Related U.S. Application Data
(60) Provisional application No. 61/477,562, filed on Apr. 20, 2011.

(51) Int. Cl. A01H 5/10

(52) U.S. Cl. CPC A01H 5/10
USPC 800/260; 800/298

(57) ABSTRACT

The present disclosure is directed to a production and cultivar system for establishing sterile, odd-ploidy Miscanthus×giganteus plantations from seed, where the seed are derived from fertile Miscanthus×giganteus parents of even but different ploidies.

### CROSS-REFERENCE TO RELATED APPLICATIONS

This application claims the benefit of U.S. Provisional Application No. 61/477,562, filed Apr. 20, 2011, which is incorporated herein in its entirety.

### FIELD OF THE INVENTION

The present disclosure relates to plant improvement.

### BACKGROUND

The perennial growth habit, low nutrient input requirement, low mineral content, and high biomass yield of members of the genus Miscanthus make it one of the most promising plant genera for biofuel production. Miscanthus biomass can be co-fired with coal in coal-burning power plants without modifications, or used as input feedstock for ethanol or other hydrocarbon production.

Miscanthus giganteus (M×g) is a particularly promising species based on its ability to produce exceptionally large biomass yield. M×g, produced by crossing Miscanthus sinensis (Msi) and Miscanthus sacchariflorus (Msa), can reach more than 3.5 m in one growing season and produce an annual dry weight yield of 25 tonnes per hectare (10 tons per acre).

Miscanthus can be planted from rhizomes obtained directly from a field of plants, or from live plants, known as “plugs,” generated in greenhouses. However, seeds are the most serviceable propagule type for scaling up plant production systems, including Miscanthus, and seed are also the most cost-effective means to establish plantations or other large-scale production fields. Plantations of M×g plants are most commonly produced from sterile triploid, clonal M×g, an expensive process that is not easily scalable but which results in a field that produces few, if any, viable seeds, or from fertile tetraploid M×g, which is a very cost-effective process but which results in a field that produces large amounts of viable seed. Sterile hybrid M×g plants propagated by rhizomes are therefore not well suited to the establishment of large-scale plantations, but plantations established cost-effectively from fertile tetraploid (or hexaploid, octaploid, etc.) M×g plants can generate a high propagule load that could lead to a substantial potential for invasiveness, depending upon the characteristics of the seed or the plants that germinate from the seed, or to a substantial stewardship cost.

There are very few agricultural systems designed in which fields are established from seed with which the resulting plants in the field do not produce much viable seed. One such example is “seedless” watermelon, a product that has seed structures that are non-fertile and which are very soft, and which therefore do not interfere with the sensory experience of eating the fruit pulp and seed. Watermelon seed for “Seedless” watermelon are more expensive to produce than seed for seeded watermelon varieties, but “Seedless” watermelon is a value-added product for which higher production seed costs can be justified.

For Miscanthus and perhaps for other biofuel plants, what is needed is a low-cost sterility system readily adapted to the establishment of a commercially practical number of seed-propagated M×g plants (that is, a plantation of seed-propagated M×g plants). It is generally expected that the mating of two different species (an interspecific cross), e.g., of Msi and Msa, would not be sufficiently productive of M×g seed of odd ploidy to provide a cost-effective seed production system. This is due to the in ability of the two species to pollinate at the same time in a seed production field. Improvements to Miscanthus lines are required through breeding and selection to generate commercially viable and largely sterile biomass varieties.

The present disclosure provides parental lines of Miscanthus spp. of even-ploidy level that, when allowed to cross pollinate under isolated controlled environments, or seed production field conditions, produce desirable (that is, commercially practical) yields of predominantly odd-ploidy M×g seed with chromosomes of both Msi and Msa. The odd-ploidy M×g seed can be grown into plants that are functionally sterile in that they produce substantially less viable pollen, have a significantly reduced seed set, and seed viability as compared, for example, to even ploidy plants or what is considered normal fertility for the species or line of which the plant is a member, such that the seed-propagated odd-ploidy M×g plants are unusable in practice as a source for germplasm. This system can be scaled-up in that it can be used in the cost-effective establishment of plantations of functionally sterile M×g plants that produce commercially valuable biomass.

### SUMMARY

Planting seed from the disclosed mating systems is produced either in a controlled environment, an experimental plot, or in a field, and by allowing for example the mating of M×g plants of even, but differing ploidy, wherein the ploidy difference may be 2, 4, 6, or 8. The resulting planting seed may segregate into frequencies of either 3×, 5× or 7× depending on the parental combinations selected for seed production. In one embodiment of the present disclosure, odd ploidy seed production is at least 8 lbs per acre, although greater yield, including 10, 15, 20, 25, 30, 35, or 40, and so on, lbs per acres are envisioned. In a preferred embodiment, odd ploidy seed is harvested from the parent in the cross that produces the highest frequency of odd ploidy seed. For example, 3× seed is derived from the 4× parent of the 4× by 2× cross in the seed production field, and 5× derived from 6× by 4× is collected from the 6× parent, and so on.

This disclosure is also directed to novel methods for producing M×g seed of a predominantly odd-ploidy, arising from the interspecific and or inter ploidy mating of Miscanthus spp. of even ploidy. The hybrid system from seed renders a cost-effective establishment of M×g plantations. The odd-ploidy plants of these plantations produce substantially less viable pollen and have a significantly reduced seed set when compared to, for example, even ploidy plants or what is considered normal fertility for the species or line of which the plant is a member, such that the seed-propagated odd-ploidy M×g plants are unusable in practice as a source for germplasm. It is generally expected that the mating of two different species, e.g., of Msi and Msa, would not be sufficiently productive of M×g seed of predominantly odd ploidy to provide a cost-effective, seed production system. Therefore, it is the object of this specification to provide a novel method for producing M×g seed of predominantly odd ploidy, in which the two parents are both, themselves, varieties of Miscanthus×giganteus, which were previously derived from the mating of Msi and Msa, at some point in their history. Since the parents of even ploidy used to create progeny of predominantly odd ploidy are of the same highly productive biomass species, M.×giganteus, seed are produced at desirable, or cost-effective, yields.

In some embodiments of the present disclosure, M×g populations and or plant genotypes are derived from the interspecific crosses of 2×Msa×2×Msi, and 4×Msa×2×Msi. The latter has produced populations containing 3× and 4× genotypes that have been selected for improving the efficiency of seed producing in the mating system. The M×g parents of even ploidy necessary for this disclosure can be derived in a number of different ways: a) by crossing diploid Msi and diploid Msa and identifying lines that when mated are fertile and contain both Msi and Msa chromosomes; b) intercrossing tetraploid Msi and tetraploid Msa and identifying lines that when mated are fertile and contain both Msi and Msa chromosomes; c) intercrossing diploid Msi and tetraploid Msa and identifying lines that when mated are fertile, are tetraploid and contain both Msi and Msa chromosomes; d) doubling the chromosome content of a sterile, triploid M×g clone to obtain a fertile, hexaploid M×g line (e.g., through colchicine treatment); e) doubling the chromosome content of a fertile, tetraploid M×g line (produced as in (b) or (c) above) to obtain a fertile, octaploid M×g line (e.g., through colchicine treatment); f) doubling the chromosome context of a sterile, pentaploid M×g line (generated by interploidy mating tetraploid and hexaploid M×g lines) to obtain a fertile, decaploid M×g line (e.g., through colchicine treatment); and so forth.

This disclosure is also directed to specific compositions, e.g., varieties or combinations of varieties of Miscanthus, including Miscanthus seed and seed-propagated progeny plants derived from said seed, wherein said seed are produced by the mating of M×g plants of even, but differing ploidy, wherein the ploidy difference may be 2, 6, or 10.

### BRIEF DESCRIPTION OF THE DRAWINGS

FIG. 1 provides a breeding diagram showing an example of a process of the present disclosure for obtaining odd ploidy number of sterile genotypes from seed for large biomass-producing Miscanthus varieties. In this example, fertile, even ploidy Miscanthus varieties (e.g., 2×, 4× or 6×) can be generated by crossing a large-stemmed Miscanthus sacchariflorus (Msa; e.g., 2× or 4×) genotype from Japan with Miscanthus sinensis (Msi; e.g., 2×). Plants in the original interspecific population crosses generate various ploidy levels that are selected upon for advanced crossing. The advanced crossing generations consist of improved plants to be used for production fields. The generated 4× and 6×Miscanthus giganteus (M×g) plants may be created by crossing 2×Msi and 4×Msa, via colchicine doubling of seedlings or using anther culture methods. Alternatively, triploid (3×) M×g plants can be produced in large amounts from 2× and 4×M×g parents that are each highly self-incompatible, but cross compatible. The ploidy of selected triploid M×g plants can be doubled using, for example, a colchicine treatment. The 4× and 6×M×g plants may then be used as parental lines for the large scale production of 5×M×g plants that are each highly self- and cross incompatible, producing little and non-viable pollen or non-viable seed. The letter “c” indicates a chromosome doubling event to achieve increase ploidy level of selected parent genotype. The letter “p” indicates ploidy segregation. Boxes indicate seed products.

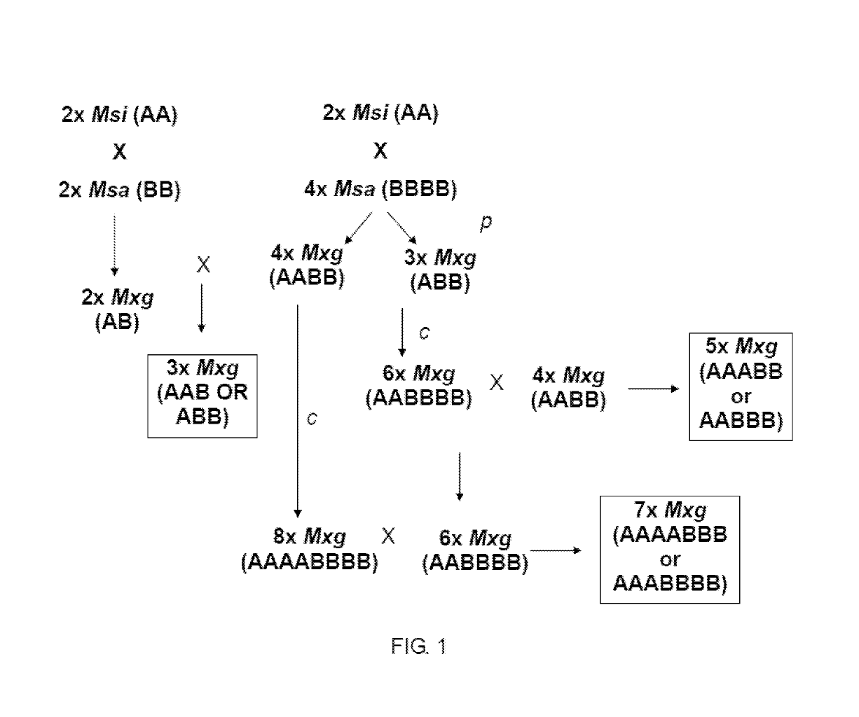

### DETAILED DESCRIPTION

The present description relates to polynucleotides and polypeptides for modifying phenotypes of plants, particularly those associated with increased abiotic stress tolerance and increased yield with respect to a control plant (for example, a wild-type plant). Throughout this disclosure, various information sources are referred to and/or are specifically incorporated. The information sources include scientific journal articles, patent documents, textbooks, and World Wide Web browser-inactive page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the instant description.

As used herein and in the appended claims, the singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

### Definitions

“Interspecific” refers to crosses or matings between individuals of different species.

“Intraspecific” refers to crosses or matings between individuals from the same species.

“Interploidy crosses” or “interploidy matings” each refer to crosses between individuals that have different total chromosomes numbers, but have even genomic ploidy number such as 2×, 4×, 6×. Interploidy matings can be accomplished for both inter and intra specific cross combinations.

“Intraploidy crosses” or “intrapolidy matings” each refer to crosses between individuals that have even and same genomic ploidy number. Intraploidy crosses can be made in inter- and intraspecific crosses.

“Cross-compatible” refers to plants that produce viable seed from outcrossing.

“Self-compatible” refers to plants that can self-pollinate, some species are able to be both cross-compatible and self-compatible, but this is relatively rare in Miscanthus spp.

“Self-incompatible” refers to a plant that is not able to self-pollinate, and is relatively common in Miscanthus spp.

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that comprise and can be used in the compositions and methods of the instant description generally include angiosperms, including plants of the class Liliopsida (monocotyledonous plants), including members of the order Poales, including members of the family Poaceae, and of the genus Miscanthus.

A “control plant” as used in the present description refers to a plant cell, seed, plant component, plant tissue, plant organ or whole plant used to compare against treated, genetically modified, or progeny plants for the purpose of identifying an enhanced phenotype in the treated, genetically modified, or progeny plants. In general, a control plant is a plant of the same line or cultivar as the treated, genetically modified, or progeny plants being tested. A suitable control plant would include a parental line used to generate treated, genetically modified, or progeny plants described herein, or genetically unaltered, wild-type, or non-transgenic plants.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to a biotic or abiotic stress, or by the observation of the expression level of a gene or genes, e.g., by employing Northern analysis, RT-PCR, microarray gene expression assays, or reporter gene expression systems, or by agricultural observations such as stress tolerance or yield. Any technique can be used to measure the amount of, comparative level of, or difference in any selected chemical compound or macromolecule in the plants, however.

“Viable” or ‘viability” refers to something that is capable of living, developing, or germinating under favorable conditions.

“Viable seed” is seed capable of germinating under favorable conditions; while “non-viable seed” is seed incapable of germinating under favorable conditions. Germination testing for seed viability is well known to those skilled in the art, see, e.g., Newmann et al., Seed Germination Testing (“Rag-Doll Test), University of Florida IFAS Extension, publication no. SS-AGR-179.

“Viable pollen” is pollen having the ability to germinate when it reaches the stigmas of flowers of its own species. Pollen viability is usually measured as the percentage of pollen grains produced that are viable. Pollen viability testing methods are well known to those skilled in the art, see, e.g., Firmage et al. (2001) Field tests for pollen viability: a comparative approach, Proc. 8^{th} Pollination Symp., Eds. P. Benedek & K. W. Richards, Acta Hort. 561:87-94. An example of measuring pollen viability is to stain collected anthers in aniline blue dye. The dye will be absorbed by the viable pollen grains, a slide is prepared and the dyed grains are counted under a microscope. Another method of measuring pollen viability uses electron particle counters. The viable pollen grains are larger than sterile grains so only particles that are of a certain size are counted.

“Yield” or “plant yield” refers to the productivity per unit area of a particular plant or plant product. For example, the yield of Miscanthus biomass is generally measured in tons per acre per season, or metric tonnes per hectare per season. Thus, yield may refer to increased biomass, increased plant growth, increased crop growth, and/or increased plant product production (including plant organs, seed, plant parts, ground plant tissue, dried plant tissue, dry biomass, wet biomass, vegetative biomass, plant cells and protoplasts, anthers, pistils, stamens, pollen, ovules, flowers, embryos, stems, buds, cotyledons, hypocotyls, roots including root tips and root hairs, rhizomes leaves, seeds, microspores and vegetative parts, whether mature or embryonic. This disclosure also relates to methods for increasing the yield of these plant parts. Yield is dependent to some extent on temperature, plant size, organ size, planting density, light, water and nutrient availability, and how the plant copes with various stresses, such as through temperature acclimation and water or nutrient use efficiency. Increased or improved yield may be measured as increased seed yield, increased plant product yield (plant products include, for example, plant tissue, including ground plant tissue, and products derived from one or more types of plant tissue), or increased vegetative yield.

When two or more plants are grown “under substantially the same environmental conditions”, they are grown in the same or very nearly the same temperatures, atmospheres (including carbon dioxide and oxygen concentrations), radiation wavelengths and flux, humidity, pathogen exposure, pest exposure, soil or growth medium quality, including pH, microflora, porosity, adsorption, absorption, nutrient or moisture levels, chemical growth enhancer levels, herbicide or pesticide levels, and to the same or very nearly the same quality, quantity and degree of the many other variables that may affect the plants' growth and development.

Depending on the appropriate context, yield” or plant yield can refer to increased plant growth, increased crop growth, increased biomass, and/or increased plant product production, and is dependent to some extent on temperature, plant size, organ size, planting density, light, water and nutrient availability, and how the plant copes with various stresses, such as through temperature acclimation and water or nutrient use efficiency. For example, Miscanthus has been reported to provide a yield of up to 18-20 tonnes of dry matter per hectare per year in one trial in Germany, but with significant variation in dry matter yield between sites in the first four years after planting (Jones and Walsh, ed. (2001) Miscanthus for Energy and Fibre, James & James, London, at page 62). Harvestable yields of Miscanthus in Europe have been reported to range from 10 to 40 tonnes of dry matter per hectare per year (Lewandowski et al, (2000) Biomass and Bioenergy 19: 209-227; Heaton et al. 2008b. supra). Heaton et al. have reported that fully established plants Miscanthus can provide typical autumn yields of dry matter ranging from 10 to 30 tonnes per hectare per year, depending on local agronomic conditions (Heaton et al. (2004) Mitigation and Adaptation Strategies for Global Change 9: 433-451). Miscanthus×giganteus autumn yields in lowland areas in Europe are typically higher than 25 tonnes per hectare per year, and Miscanthus×giganteus could provide a hypothetical yield of 27-44 tonnes of dry matter per hectare per year with a mean yield of 33 tonnes of dry matter per hectare per year in Illinois (Heaton et al. (2004) supra). Miscanthus×giganteus can thus yield, under various conditions of growth, biomass of at least 10, at least 15, at least 20, at least 25, at least 27, at least 30, at least 33, at least 35, at least 40, at least 44 tonnes or more of dry matter per hectare per year. It is expected that the poorly fertile or sterile, seed-propagated varieties of Miscanthus described herein can produce similar biomass yields, ranging from, for example, at least 70%, at least 75%, at least 80%, at least 85%, at least 90%, at least 95%, at least 100%, at least 105%, at least 110%, at least 115%, at least 120%, at least 125% or more of the biomass yield of a control sterile triploid M×g crop (e.g., the M×g ‘Illinois’ clone) when at substantially the same stage of seedling development and when grown under substantially the same, or the same, environmental conditions. In other words, the instantly disclosed poorly fertile or sterile, seed-propagated M×g varieties are expected to yield at least 75% to at least 125% or more of 10 to 44 tonnes or more of dry matter per hectare per year. An ‘M.×giganteus’ (‘M×g’) plant is a hybrid of Miscanthus sinensis and Miscanthus sacchariflorus. Quite often, hybrids obtained by crossing related species result in heterosis, or hybrid, and M×g is no exception. M×g is a thus useful species for producing biomass for production of biofuels or renewable electricity. For example, the M.×giganteus ‘Illinois clone’ (M×g; 2n=3x=57) is a sterile triploid hybrid of a diploid Msi (2n=2x=38) and a tetraploid Msa (2n=4x=76). The M×g ‘Illinois clone’ generally produces high biomass relative to other Miscanthus plants, has relatively high nitrogen use efficiency and is able to grow well on low nutrient or set-aside land without intensive fertilization.

Other M×g genotypes and methods for generating these various genotypes, including fertile parental lines for the generation of sterile, odd ploidy M×g lines, also exist and are envisioned, including by, for example:
a) crossing diploid Msi and diploid Msa and identifying lines that when mated are fertile and contain both Msi and Msa chromosomes;
b) crossing tetraploid Msi and tetraploid Msa and identifying lines that when mated are fertile and contain both Msi and Msa chromosomes;
c) doubling the chromosome content of a sterile, triploid M×g clone to obtain a fertile, hexaploid M×g line (e.g., through colchicine treatment);
d) doubling the chromosome content of a fertile, tetraploid M×g line (produced as in (b) or (c) above) to obtain a fertile, octaploid M×g line (e.g., through colchicine treatment); or
e) doubling the chromosome context of a sterile, pentaploid M×g line (generated by mating tetraploid and hexaploid M×g lines) to obtain a fertile, decaploid M×g line (e.g., through colchicine treatment); and so forth.

“Functional sterility” (or “phenotypic sterility”) refers to a level of fertility that is sufficiently low, compared to what is considered normal fertility for the species or line of which the plant is a member, to render the plant unusable in practice as a source for germplasm. One indicator of functional sterility is a significantly reduced seed set relative to the “wild type” or a non-variant plant of the pertinent species or line. In a given instance, a seed yield that is significantly less than average for a variant plant (for example, an odd-ploidy M×g plant) could be deemed indicative of functional sterility. The threshold indication of functional sterility could be a low as or less than, for example, 10% or 5%, 1% of average, or 0.2%, 0.1%, or 0.01% of the average of a control plant such as a wild-type plant a non-variant plant, or an even-ploidy plant. Another indicator of functional sterility is the continued production of a high percentage of abortive, nonfunctional and/or non-viable pollen grains when variant plant material is used in a large number of outcrosses. What is “large” in this context would depend on what is considered normal in the context of pollen output for a control plant, for example, the species or line representing the wild type of the variant in question or an even ploidy parent plant. In general, an incidence among all pollen produced of between 1% to 10% (inclusive), or less than 5%, or less than 1%, or less than 0.2%, or less than 0.1%, or between 0.01% to 0.1% (inclusive), or less than 0.01% functional pollen grains is indicative of functional sterility, since these low levels of functional pollen dramatically decrease the likelihood of a functional pollen grain encountering an appropriate stigma. In this regard, “functional” pollen is pollen that will fertilize an egg cell and produce a viable embryo when the pollen is used in a cross under conditions that are normal for the species involved, and availability of functional pollen is limited by the diminished ability of the variant to produce it. Thus, a variant plant (for example, an odd-ploidy M×g plant) that possesses a level of fertility that is less than 10%, or less than 5%, less than 1%, or less than 0.2%, or less than 0.1%, or less than 0.01% of what is expected of a control plant (e.g., a wild-type plant, a non-variant, or an even ploidy parent plant) is an indication that the variant plant is functionally sterile (see U.S. Pat. No. 5,049,503).

“Planting density” refers to the number of plants that can be grown per acre. For crop species, planting or population density varies from a crop to a crop, from one growing region to another, and from year to year. Using corn as an example, the average prevailing density in 2000 was in the range of 20,000-25,000 plants per acre in Missouri, USA. A desirable higher population density (a measure of yield) would be at least 22,000 plants per acre, and a more desirable higher population density would be at least 28,000 plants per acre, more preferably at least 34,000 plants per acre, and most preferably at least 40,000 plants per acre. The average prevailing densities per acre of a few other examples of crop plants in the USA in the year 2000 were: wheat 1,000,000-1,500,000; rice 650,000-900,000; soybean 150,000-200,000, canola 260,000-350,000, sunflower 17,000-23,000 and cotton 28,000-55,000 plants per acre (Cheikh et al. (2003) U.S. Patent Application No. 20030101479). For Miscanthus, a typical initial planting density is 10,000 plants per hectare (Scurlock (1999) Miscanthus: A Review of European Experience with a Novel Energy Crop, U.S. Department of Energy, Publ. ORNL/TM-13732, at page 6). A desirable higher population density for each of these examples, as well as other valuable species of plants, including Miscanthus, would be at least 5%, or at least 10%, or at least 15%, or at least 20%, or at least 25%, or higher, than the average prevailing density or yield.

Plant breeders have historically used a various breeding, hybridization and selection techniques to create improved plant types. “Population improvement” can be used for the improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Increased uniformity is achieved in such populations through rigorous selection pressure, but is not as rapid as for self-pollinating species. In addition, trueness-to-type in an open-pollinated cultivar is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Population improvement involving multiple species cross combinations utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations.

In general, Miscanthus is an out-crossing, wind-pollinated species. See, e.g., Deuter, P. D. (2000) Breeding approaches to improvement of yield and quality in Miscanthus grown in Europe, In: European Miscanthus improvement—final Report September 2000, I. Lewandowski & J. C. Clifton-Brown (Eds.), pp. 28-52, Institute of Crop Production and Grassland Research, University of Hohenheim, Stuttgart, Germany. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic cultivar attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

In “mass selection,” desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

A “synthetic” cultivar is produced by crossing inter se a number of genotypes selected for general combining ability in all possible hybrid combinations, with subsequent maintenance of the cultivar by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or toperosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and may thus be considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that can be cohybridized to generate a synthetic allopolyploid cultivar vary widely. In practice, numbers of parental lines range from 10 to several hundred, with 100-200 being the average. Broad based synthetics formed from 100 or more clones would be expected to be more stable during seed multiplication than narrow based synthetics.

A “hybrid” is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of hybrid production processes, see, e.g., Wright, Commercial Hybrid Seed Production 8:161-176; and, more particularly, for a detailed discussion of methods for the natural/artificial hybridization and self-pollination of various representative grass species, see, e.g., Hovin, Cool-Season Grasses 18:285-298; In: Hybridization of Crop Plants (1980) American Society of Agronomy and Crop Science Society of America, Publishers, Madison, Wis.

Commercial Miscanthus seed may be provided either in a synthetic cultivar or a hybrid cultivar. Commercial production of synthetic varieties may include a breeder seed production stage, a foundation seed production stage, a registered seed production stage and a certified seed production stage. Hybrid cultivar seed production may involve up to three stages including a breeder seed production stage, a foundation seed production stage and a certified seed production stage.

The ability to produce and plant seed of biomass-yielding species has significant practical and financial implications. For example, the cost and effort of seed generation is significantly less than that associated with seedlings or plugs containing rhizomes, and can also result in improved volume and throughput. Sowing seed derived from Miscanthus species, for example, will generally cost less than the costs that would be associated with sowing plugs or seedlings. Farmers can thus plant more seeds with less cost, and with less effort, which allows for more plants to be seeded per unit area. The resulting initial higher planting density would bring about reduced costs per unit mass. As there is a significant positive correlation between initial planting density and yield in the first few years of growth (Jones and Walsh, ed., 2001, supra, at page 62), higher planting densities may also allow the farmer to produce for a commercially serviceable crop at the end of the first year of growth and better profit margins for the first few years after planting.

Miscanthus varieties have been developed through a combination of breeding and selection processes, the latter used to select for advantageous traits including, but not limited to, fertility, improved biomass, increased vigor, increased vigor at the seedling stage, increased water deficit tolerance, and greater tiller density. These improved characteristics were shown to be heritable, and it is expected that further improvements may be made with these varieties.

### Description of the Specific Embodiments

Miscanthus plantations are designed to produce maximum levels of high-quality biomass per acre. Seed are not needed in a production field, but are the best propagules for scaling Miscanthus production systems and the most cost-effective way of producing propagules for plantation establishment and for establishing those plantations. There are a number of possible ways for plants sown from seed to result in plants that do not produce seed in a production field. One such way is to generate commercial seed to be used by a grower by filed crossing two parents of even ploidy (e.g., 2×, 4×, 6×, 8×), with the ploidy levels differing by only 2 chromosome copies (e.g., 2× and 4×; 4× and 6×). Plants that have an odd ploidy are generally sterile by virtue of the inability for effective pairing of the odd-numbered genomes.

This present specification describes ways to create fields of plants that contain a vast majority of sterile M×g plants of odd-ploidy, established from seeds created by the crossing of two different Miscanthus parents of differing, even ploidies as described above. With the goal of establishing M×g plantations through seed, preferably with few viable seeds, that is, less than 5%, or less than 1%, or less than 0.2%, or less than 0.1%, or less than 0.01% of viable seed in the population compared to standard, fertile tetraploid M×g (FTMG), both parents need to be of the same M×g species. Generally, present experience suggests that it is cost prohibitive to produce M×g seed from parents of Msi and Msa directly due very low fertility. Selection for superior parents is needed for biomass efficiency as well as the ability to be cross compatible in the seed production field. Cross compatibility in this example is for the anthesis (fertilization)) timing for parents involved in the field cross as well as other recombination effects on biomass traits.

Therefore, the present disclosure pertains to the creation of odd-ploidy M×g seed by crossing two M×g parents of differing, even ploidy (or more parents so long as the parents with the same ploidy level are self-incompatible, but compatible with the M×g parent of differing ploidy level). One of the keys to this disclosure is the creation of M×g even-ploidy parents with good combining ability for biomass yield, and with good fertility when crossed to each other. Applicants have created diploid (2×) M×g through the controlled cross of specific 2×Msi and 2×Msa parents, tetraploid (4×) M×g through the controlled cross of specific 2×Msi and 4×Msa parents, hexaploid (6×) M×g through the chromosome doubling of 3×M×g (‘Illinois’ clone or ‘MBS 7001’; for more description of the latter, see plant patent application Ser. No. 12/387,444, filed 1 May 2009, herein incorporated by reference) and through embryogenic culture of anthers to create callus derived from pollen, followed by chromosome doubling.

The present disclosure describes the production of odd ploidy (e.g., triploid (3×), pentaploid (5) or septaploid (7), etc.) M×g seed in large amounts from even ploidy (e.g., 2× and 4×, or 4× and 6×, or 6× and 8×, etc.) M×g parents that are each highly self-incompatible but cross compatible, and the production of triploid, pentaploid or septaploid, etc., (3×, 5×, 7×, etc.) M×g seed.

Table 1 lists interspecific and intraspecific crosses and ploidy manipulation examples of Msa, Msi, and M×g species used for producing odd ploidy seeded Miscanthus, and illustrates how deriving an odd ploidy genotype or population of seed propagated production biomass field can occur through both inter- and intraspecific cross combinations. Intra-specific odd ploidy parents are derived via chromosome doubling and or through anther culture techniques. Products derived from the odd ploidy mating system can be either a single genotype or a line population that segregates for odd and even ploidy. The desired line product would have 98% or better of all odd ploidy genotypes from seed in the line. Lines less that 98% odd ploidy are then used for selection of superior odd ploidy single genotypes.

TABLE 1
Miscanthus inter- and intraspecific and/or ploidy cross combinations
used to derive parents and odd ploidy seed products

| Interspecific Matings | | | Intraspecific Matings | | |
|---|---|---|---|---|---|
| [2xMsi × | → | Interspecific & | [4x Msi × | → | Intraspecific & |
| 2xMsa] | | Intraploidy (all | 2xMsi] | | Interploidy via |
| | | 2x) | | | chromosome |
| [4x Msa × | → | Interspecific & | | | doubling of |
| 2xMsi] | | Interploidy | | | 2x Msi (2x, 3x, |
| | | (2x, 3x, 4x | | | 4x segregants) |
| | | segregants) | [4x Mxg × | → | Intraspecific & |
| [4x Mxg × | → | Interspecific & | 6xMxg] | | Interploidy via |
| 2xMsi] | | Interploidy | | | chromosome |
| | | (2x, 3x, 4x | | | doubling of |
| | | segregants) | | | 3x Mxg (4x, |
| [4x Mxg × | → | Interspecific & | | | 5x, 6x |
| (2xMsa*2xMSi)] | | Interploidy | | | segregants) |
| | | (2x, 3x, 4x | [8x Mxg × | → | Intraspecific & |
| | | segregants) | 6x Mxg] | | Interploidy via |
| | | | | | chromosome |
| | | | | | doubling of |
| | | | | | 4x Mxg and |
| | | | | | 3xMxg (4x, 6x, |
| | | | | | 7x segregants) |

Other combinations of M×g parents of different even ploidies can be envisioned (see Example III). One of the critical features of the present disclosure is the low fertility/near sterility of the plants resulting from sowing the triploid or pentaploid M×g seed produced as described above. In comparison with the fertility of two ETMG plants of differing incompatibility groups, such as ‘MBS 7002’ and ‘MBS 1001’, or ‘MBS 1001’ and ‘MBS 1002’ (each produced clonally; see U.S. Plant Pat. No. 22,047, U.S. Plant Pat. No. 22,127, U.S. Plant patent application Ser. No. 13/067,964, and publicly-available U.S. patent application Ser. No. 12/387,429 for more description of each of these lines), the resulting seed yield from triploid or pentaploid M×g derived directly from seed is less than 5%, or less than 1%, or less than 0.5%, preferably less than 0.2%, or less than 0.1%, more preferably less than 0.01%, and more preferably less than 0.001%.

The present disclosure differs in very significant ways from existing technology for establishing Miscanthus plantations. Today's plantations are either established from sterile triploid, clonal M×g, an expensive process that is not easily scalable, yielding a field that produces few, if any, viable seeds, or from fertile tetraploid M×g, a very cost-effective process, but which yields a field that produces large amounts of viable seed. This present disclosure offers the combination of the best of both alternatives, a low-cost, seed-propagated, high-yielding Miscanthus establishment process in a largely sterile stand or plantation.

### EXAMPLES

It is to be understood that this description is not limited to the particular devices, machines, materials and methods described. Although particular embodiments are described, equivalent embodiments may be used to practice the claims.

The specification, now being generally described, will be more readily understood by reference to the following examples, which are included merely for purposes of illustration of certain aspects and embodiments of the present description and are not intended to limit the claims or description. It will be recognized by one of skill in the art that a polypeptide that is associated with a particular first trait may also be associated with at least one other, unrelated and inherent second trait which was not predicted by the first trait.

### Example I

### Preparation of Fertile M×g Parents and Creation of a Seed Production Field

Seeds created by crossing two different M×g parents of differing, even ploidies may be used to produce parental lines that serve in breeding programs for the production of sterile M×g plants of odd-ploidy. For example, fertile, even ploidy Miscanthus varieties are generated by crossing a large-stemmed Msa genotype from Japan with Msi plants as pollen donors. Triploid (3×) M×g seed can thus be produced in large amounts from 2× and 4×M×g parents that are each highly self-incompatible but highly cross-compatible, as can the production of pentaploid (5×) M×g seed in large amounts be produced from 4× and 6×M×g parents that are each highly self-incompatible but highly cross-compatible. One of the important features of this disclosure is the low fertility/near sterility of the progeny plants resulting from sowing the triploid or pentaploid M×g seed produced as described herein. In comparison with the cross-compatibility of two FTMG plants of differing incompatibility groups, such as ‘MBS 7002’ and ‘MBS 1001’, or ‘MBS 1001’ and ‘MBS 1002’ (each produced clonally; see U.S. Plant Pat. No. 22,047, U.S. Plant Pat. No. 22,127, U.S. Plant patent application Ser. No. 13/067,964, and publicly-available U.S. patent application Ser. No. 12/387,429 for more description of these lines), the resulting yield of seed from seed-propagated odd ploidy (for example, triploid or pentaploid M×g,) that can produce fertile plants is less than 5%, or less than 1%., or less than 0.5%, preferably less than 0.2%, or less than 0.1%, more preferably less than 0.01%, and more preferably less than 0.001%.

Optionally, these parental lines are selected for strong self-incompatibility and/or efficient cross compatibility. Self-incompatibility, a pollen-rejection system in which pollen recognition by the stigma is determined by tightly linked and co-evolving alleles of the S-locus receptor kinase (SRK) and its S-locus cysteine-rich ligand (SCR), prevents inbreeding in flowering plants (Boggs et al. (2009) PLoS Genet. 5: e1000426). In contrast, cross compatibility refers to sexual compatibility between plants. From the crossing of these parental lines, seedlings are obtained and planted in a controlled environment, an experimental plot or field.

Varieties that have been created to date include diploid (2×) M×g through the controlled cross of specific 2×Msi and 2×Msa parents, tetraploid (4×) M×g through the controlled cross of specific 2×Msi and 4×Msa parents, hexaploid (6×) M×g through the chromosome doubling of 3×M×g (‘Illinois’ clone or ‘MBS 7001’) and through embryogenic culture of anthers to create callus derived from pollen, followed by chromosome doubling to achieve hexaploid (6×) M×g varieties. Selection of high-biomass, even ploidy varieties are then made. These varieties may be selected for good combining ability for biomass yield, strong self-incompatibility and/or efficient cross compatibility (i.e., good fertility when crossed to each other). Of particular interest as potential parental lines are Miscanthus plants of differing ploidy, wherein the ploidy difference is 2, 6, 8, or 10.

Control plants used as comparators of biomass yield, size, vigor, or other traits may include Miscanthus×giganteus (M×g) ‘Giant Miscanthus’ or the M×g ‘Illinois’ clone, which is well known and readily available to the public. M×g ‘Illinois’ clone is described in a number of publications, including Greef et Deu ex. Hodkinson et Renvoize; Heaton et al. (2008a) Curr. Opin. Biotechnol. 19: 202-209 and Heaton et al. (2008b) Global Change Biol. 14: 2000-2014. M×g ‘Illinois’ clone is commercially available from a number of sources, including but not limited to:

Speedling, Inc.

P.O. Box 7220

Sun City, Fla. 33586-7220

New Energy Farms Ltd

209 Erie Road North

Leamington, Ontario N8H 3A5

Earth Sense Energy USA

PO Box 14705

San Luis Obispo, Calif. 93406

South Farms

University of Illinois 1301 W. Gregory Drive

Urbana, Ill. 61801

Victoriana Nursery Gardens,

Challock, Nr Ashford, Ky.

TN25 4DG, England, UK

### Example II

### Propagation of Fertile, Even Ploidy Miscanthus Parental Lines

Fertile, even ploidy Miscanthus parental lines are propagated from rhizomes, meristems, nodes, or other vegetative tissues in which the genetic composition of the propagated plants is the same as the plants from which the tissues are derived (i.e. these plants are propagated by cloning). The cloning of parent lines is desirable because it maintains the genetic identity of the parental lines, and it overcomes the barrier imposed by the largely self-incompatible reproductive biology of Miscanthus spp. Self incompatibility limits the ability to propagate parental lines by seeds. A field of a pure stand of such clonally derived parents produces very few seeds.

Methods for the cloning of Miscanthus have long been known in the art as demonstrated by, for example, Nielsen 1987. Tidsskr. Planteavl. 91: 361-368. Propagation can be done ex-vitro (e.g. by division of rhizomes) or in vitro. Multiple in vitro methods for Miscanthus propagation have been described including methods utilizing axillary shoots (Nielsen et al. 1995. Plant Cell Tiss. Org. Cult. 41: 165-170), and methods which involve the initiation and propagation of plant callus (Petersen 1997. Plant Cell Tiss. Org. Cult. 49: 137-140).

### Example III

### Various Crosses that Produce M×g Plants of Various Predominantly Odd Ploidies

The crossing of pairs of parental lines of even ploidy (for example, differing in ploidy by 2×, where x is an odd number; e.g., when x=1, 3, 5, etc., or where the pairs of parental lines have ploidies of 2 and 4, or 4 and 6; or 2 and 8, etc.), wherein the lines of even ploidy are shown to be largely self-incompatible by testing for seed production on inflorescences when the inflorescences are “bagged” to protect from pollen sources other than those of the self-plant, is used to produce seed of predominantly odd ploidy. The mating of plants derived clonally from an ‘Amuri’ line, wherein ‘Amuri’ lines may be generated by intercrossing diploid Msi and diploid Msa, with plants derived clonally from a fertile tetraploid line, ‘MBS 7002’ (also referred to as ‘MBX-004’), as described in U.S. Plant Pat. No. 22,047, issued 26 Jul. 2011, which is herein incorporated by reference, results in the formation of predominantly triploid seed with a seed yield acceptable to provide for cost-effective seed production and plantation establishment. The planting system may use an alternating two row pattern of 2 rows 2× as the male, 2 rows 4× as the female, etc. Only seed produced by the 4× female will be harvested. The 4× female may be ‘MBS 1001’ (described in U.S. Plant patent application Ser. No. 13/067,964 and publicly-available U.S. patent application Ser. No. 12/387,429; also referred to as ‘MBX-005’) and the 2× male may be ‘MBS 0010’ (described in publicly-available U.S. patent application Ser. No. 12/387,429). A representative sample of the marketable seed (also referred to as “clean seed”, or seed that has been cleaned of all chaff, other inert material, broken seed, light seed and small seed, wherein the marketable seed is generally the seed that is to be used for planting) from the harvest may then be tested for percent ploidy levels (the percentage of each ploidy level found in the marketable seed; for example, when 3× is the expected genotype, there may be a low level of 2× or 4× fertile genotypes produced).

In one embodiment of the present disclosure, the mating of the first even ploidy M×g plant and a second even but different ploidy M×g plant produces a percentage of viable (that is, can be grown into a progeny M×g plant) odd ploidy seed of at least 5% for derivation of odd ploidy genotypes from the mating system. Single genotypes have value for analysis of sterility effects as well as biomass potential and as a final vegetative propagule.

The preferred percentage for a seed propagated population is at least 5%, at least 7.5%, at least 10%, at least 12.5%, at least 15%, at least 17.5%, at least 20%, at least 22.5%, at least 25%, at least 27.5%, at least 30%, at least 32.5%, at least 35%, at least 37.5%, at least 40%, at least 42.5%, at least 45%, at least 47.5%, at least 50%, at least 52.5%, at least 55%, at least 57.5%, at least 60%, at least 62.5%, at least 65%, at least 67.5%, at least 70%, at least 72.5%, at least 75%, at least 77.5%, at least 80%, at least 82.5%, at least 85%, at least 87.5%, at least 90%, at least 92.5%, at least 95%, at least 99%, at least 99%, at least 99.9%, to about 100% odd ploidy of total seed produced so that the bulk of the population has predictable, and uniform incompatibility for reducing seed set and seed viability in the biomass field. Preferably, the progeny plants that are seed-established in the field produce “few or no viable seed”, that is, the aforementioned even ploidy×different even ploidy cross results in seed that are grown into progeny plants that then produce seed of which fewer than 5%, or less than 1%, or less than 0.2%, or less than 0.1%, or less than 0.01% are viable.

Thus far the odd ploidy mating system has provided seeded populations that have segregated for odd ploidy. One example involves a particular line genotype known as ‘MBS 7001’ (also referred to as ‘MBX-002’ or ‘Nagara’) which has produced less than 1% seed set compared to its sister line genotypes that have produced as much as 75% seed set under open field pollinated conditions.

The ploidy of expected predominantly triploid progeny from a sample population of the ‘MBS 0010’×‘MBS 1001’ cross are verified by flow cytometry using a flow cytometer such as a Partec CyFlow® Ploidy Analyser and any of several buffer combinations known in the art.

The interploidy mating of plants derived clonally from a fertile tetraploid line, ‘MBS 7002’, as described in U.S. Plant Pat. No. 22,047, supra, with plants derived by colchicine treatment of sterile, triploid ‘Nagara’ (also referred to as ‘MBX-002’; ‘Nagara’ is described in publicly-available U.S. patent application Ser. No. 12/387,429, which is herein incorporated by reference), to give fertile, hexaploid M×g genotype ‘J130219’ (also referred to as ‘00m0007001CD1’), results in the formation of predominantly pentaploid seed with a seed yield acceptable to provide for cost-effective seed production and plantation establishment. This process of odd ploidy segregation verification is still being evaluated in the field.

The ploidy of expected predominantly pentaploid progeny from 4×‘MBS 7002’×6×‘J130219’ crosses are verified by flow cytometry. Additional crosses between 2× and 4× genotypes derived from the MBS mating system have generated populations that vary from 0% to 25% odd ploidy, indicating selection potential for appropriate matings of 2×X 4× or 4×X 6× matings, and so on. These populations have been developed and are currently being evaluated for higher frequency results of the particular matings for odd ploidy genetic expression. New populations are being derived that involve combinations of 6× and 8×, and using 4× genotypes from chromosome doubling of 2×M. sinensis and 2× ‘Amuri’ (population derived from 2×M. sacchariflorus×2×M. sinensis) to be crossed with plants having 6× chromosomes doubled from 3× genotypes.

### Example IV

### Improved Yield Produced by Sterile, Seed Propagated Varieties

Miscanthus varieties are expected to develop significantly more biomass than many other plants considered as feedstock candidates, including switchgrass. For example, in an experimental field trial conducted in ‘Illinois,’ Miscanthus×giganteus yielded approximately twice the biomass as switchgrass.

This disclosure also relates to the use of these plant parts for regenerating plants. The plant parts (e.g., rhizomes or other plant parts), seeds, cells, tissue culture, etc. may be used to regenerate plants having substantially all the improved morphological and physiological characteristics of the selected Miscanthus varieties described herein.

By the use of the methods described herein, a population of odd ploidy, poorly fertile or sterile M×g plants is produced. A stand or population of this odd ploidy, poorly fertile or sterile M×g may be produced or propagated by seed, although other means of propagation, such as with rhizomes, meristems, nodes, other vegetative tissues, or other asexual reproductive means, may also be used to expand the population. One important distinction between the seed-propagated M×g plants and the M×g ‘Illinois’ plants is that the former may obviously be established by seed, whereas the latter is established with seedlings, plugs containing rhizomes, or other asexual reproductive means.

It is expected that a stand of the odd ploidy, poorly fertile or sterile, seed-propagated, M×g plants will produce a biomass yield similar to that which may be produced by the same number of M×g ‘Illinois’ plants, when the seed-propagated M×g plants and the M×g ‘Illinois’ plants are grown under substantially the same environmental conditions. The biomass yield of the seed-propagated M×g plants may be at least 70% of the biomass yield produced by the equal number of M×g ‘Illinois’ plants, or at least 75%, at least 80%, at least 85%, at least 90%, at least 95%, at least 100%, at least 105%, at least 110%, at least 115%, at least 120%, at least 125% or more of the biomass yield of the M×g ‘Illinois’ plants, when the population of odd ploidy, seed propagated M×g plants and the M×g ‘Illinois’ plants are grown under substantially the same environmental conditions.

All publications and patent applications mentioned in this specification are herein incorporated by reference to the same extent as if each individual publication or patent application was specifically and individually indicated to be incorporated by reference.

The present disclosure is not limited by the specific embodiments described herein. The specification now being fully described, it will be apparent to one of ordinary skill in the art that many changes and modifications can be made thereto without departing from the spirit or scope of the appended claims.

Modifications that become apparent from the foregoing description and accompanying figures fall within the scope of the claims. Further aspects of this specification include the following numbered embodiments:
Embodiment 1. A method for producing a plurality of seed-propagated Miscanthus×giganteus (M×g) plants that produce few or no viable seeds, the method comprising:
   (a) providing a first M×g plant with an even ploidy number and a second M×g plant having a different even ploidy number from that of the first M×g plant; and
   (b) mating the first M×g plant and the second M×g plant; and
   (c) producing viable, odd ploidy M×g seed from the mated first M×g plant and the second M×g plant; and
   (d) growing a plurality of odd ploidy seed-propagated M×g progeny plants from the viable, odd ploidy M×g seed;
      wherein the odd ploidy seed-propagated M×g progeny plants produce few or no viable seeds.
Embodiment 2. The method of embodiment 1, wherein at least 10% of total seed produced by the mating of the first M×g plant and the second M×g plant are odd ploidy and viable.
Embodiment 3. The method of embodiment 1, wherein or less than 10%, or less than 5%, or less than 2.5%, or less than 1%, or less than 0.2%, or less than 0.1%, or less than 0.01%, of the total seed produced from the odd ploidy seed-propagated M×g progeny plants are viable.
Embodiment 4. The method of embodiment 1, wherein the mating of the first M×g plant and the second M×g plant produces a yield of at least 8 pounds per acre of viable, odd ploidy M×g seed.
Embodiment 5. The method of embodiment 1, wherein the odd ploidy seed-propagated M×g progeny plants are pentaploid.
Embodiment 6. The method of embodiment 1, wherein the odd ploidy seed-propagated M×g progeny plants are triploid.
Embodiment 7. The method of embodiment 1, wherein the ploidy number difference between the first M×g plant and the second M×g plant is 2, 6, or 10.
Embodiment 8. The method of embodiment 1, wherein the plurality of seed-propagated M×g progeny plants produces a biomass yield of at least 80% of the biomass yield produced by an equal number of M×g ‘Illinois’ clone plants when the progeny plants and the M×g ‘Illinois’ clone plants are grown under substantially the same environmental conditions.
Embodiment 9. The method of embodiment 8, wherein the biomass yield of the seed-propagated M×g progeny plants is at least 100% of the biomass yield produced by the equal number of M×g ‘Illinois’ clone plants when the seed-propagated M×g progeny plants and the M×g ‘Illinois’ clone plants are grown under substantially the same environmental conditions.
Embodiment 10. The method of embodiment 1, wherein the first M×g plant and the second M×g plant are selected for self-incompatibility and cross-compatibility.
Embodiment 11. A viable, odd ploidy M×g seed produced by the mating of the first M×g plant of embodiment 1 and the second M×g plant of embodiment 1.
Embodiment 12. An odd ploidy, seed-propagated M×g progeny plant that produces few or no viable seeds, wherein the odd ploidy M×g progeny plant is grown from the viable, odd ploidy M×g seed of embodiment 11.
Embodiment 13. A method for producing a viable M×g seed having an odd ploidy number, the method comprising:
   (a) providing a first M×g plant with an even ploidy number and a second M×g plant having a different even ploidy number from that of the first M×g plant;
   (b) mating the first M×g plant and the second M×g plant; and
   (c) producing a viable M×g seed having an odd ploidy number from the mated first M×g plant and the second M×g plant.
Embodiment 14. The method of embodiment 13, wherein the mating of the first M×g plant and the second M×g plant produces a percentage of viable odd ploidy seed of at least 10% of total seed produced.
Embodiment 15. The method of embodiment 13, wherein the mating of the first M×g plant and the second M×g plant produces a yield of at least 8 pounds per acre of viable odd ploidy seed.
Embodiment 16. The method of embodiment 13, wherein the ploidy number difference between the first M×g plant and the second M×g plant is 2, 6, or 10.
Embodiment 17. The method of embodiment 13, wherein the first M×g plant and the second M×g plant are selected for self-incompatibility and cross-compatibility.
Embodiment 18. A viable, odd ploidy M×g seed produced by the mating of the first M×g plant of embodiment 13 and the second M×g plant of embodiment 13.
Embodiment 19. An odd ploidy, seed-propagated M×g progeny plant that produces few or no viable seeds, wherein the odd ploidy M×g progeny plant is grown from the viable odd ploidy M×g seed of embodiment 18.
Embodiment 20. The odd ploidy, seed-propagated M×g progeny plant of embodiment 19, wherein or less than 10%, or less than 5%, or less than 2.5%, or less than 1%, or less than 0.2%, or less than 0.1%, or less than 0.01%, of the seeds produced from the odd ploidy seed-propagated M×g progeny plants are viable.
Embodiment 21. A method of biofuel production comprising using feedstock for said biofuel production, wherein said feedstock comprises plant biomass produced from a plurality of odd ploidy seed-propagated M×g progeny plants produced by:
   (a) providing a first M×g plant with an even ploidy number and a second M×g plant having a different even ploidy number from that of the first M×g plant;
   (b) mating the first M×g plant and the second M×g plant;
   (c) producing odd ploidy, viable seed from the mated first M×g plant and the second M×g plant; and
   (d) growing a plurality of odd ploidy seed-propagated M×g progeny plants from the viable seed to produce a population of M×g progeny plants that comprises said feedstock, wherein the progeny plants produce few or no viable seeds.
Embodiment 22. The method of embodiment 21, wherein the mating of the first M×g plant and the second M×g plant produces a yield of at least 8 pounds per acre of viable odd ploidy seed.
Embodiment 23. The method of embodiment 21, wherein the plant biomass produced from the plurality of odd ploidy seed-propagated M×g progeny plants is at least 5 tons per acre.
Embodiment 24. The method of embodiment 21, wherein the plurality of seed-propagated M×g progeny plants produces a biomass yield of at least 80% of the biomass yield produced by an equal number of M×g ‘Illinois’ clone plants when the progeny plants and the M×g ‘Illinois’ clone plants are grown under substantially the same environmental conditions.
Embodiment 25. The method of embodiment 24, wherein the biomass yield of the seed-propagated M×g progeny plants is at least 100% of the biomass yield produced by the equal number of M×g ‘Illinois’ clone plants when the seed-propagated M×g progeny plants and the M×g ‘Illinois’ clone plants are grown under substantially the same environmental conditions.

What is claimed is:

1. A method for producing a plurality of seed-propagated Miscanthus×giganteus (M×g) plants that produce few or no viable seeds, the method comprising:
(a) providing a first M×g plant with an even ploidy number and a second M×g plant having a different even ploidy number from that of the first M×g plant; and
(b) mating the first M×g plant and the second M×g plant; and
(c) producing viable, odd ploidy M×g seed from the mated first M×g plant and the second M×g plant; and
(d) growing a plurality of odd ploidy seed-propagated M×g progeny plants from the viable, odd ploidy M×g seed;
wherein the odd ploidy seed-propagated M×g progeny plants produce few or no viable seeds.

2. The method of claim 1, wherein at least 10% of total seed produced by the mating of the first M×g plant and the second M×g plant are odd ploidy and viable.

3. The method of claim 1, wherein less than 10%, or less than 5%, or less than 2.5%, or less than 1%, or less than 0.2%, or less than 0.1%, or less than 0.01%, of the total seed produced from the odd ploidy seed-propagated M×g progeny plants are viable.

4. The method of claim 1, wherein the mating of the first M×g plant and the second M×g plant produces a yield of at least 8 pounds of viable, odd ploidy M×g seed per acre.

5. The method of claim 1, wherein the odd ploidy seed-propagated M×g progeny plants are triploid (3×).

6. The method of claim 1, wherein the odd ploidy seed-propagated M×g progeny plants are pentaploid (5×).

7. The method of claim 1, wherein the odd ploidy seed-propagated M×g progeny plants are septaploid (7×).

8. The method of claim 1, wherein the ploidy number difference between the first M×g plant and the second M×g plant is 2, 6, 8, or 10.

9. The method of claim 1, wherein the plurality of seed-propagated M×g progeny plants produces a biomass yield of at least 80% of the biomass yield produced by an equal number of M×g ‘Illinois’ clone plants when the progeny plants and the M×g ‘Illinois’ clone plants are grown under substantially the same environmental conditions.

10. The method of claim 9, wherein the biomass yield of the seed-propagated M×g progeny plants is at least 100% of the biomass yield produced by the equal number of M×g ‘Illinois’ clone plants when the seed-propagated M×g progeny plants and the M×g ‘Illinois’ clone plants are grown under substantially the same environmental conditions.

11. The method of claim 1, wherein the first M×g plant and the second M×g plant are selected for self-incompatibility and cross-compatibility.

12. A viable, odd ploidy M×g seed produced by the crossing of the first M×g plant of claim 1 and the second M×g plant of claim 1.

13. An odd ploidy, seed-propagated M×g progeny plant that produces few or no viable seeds, wherein the odd ploidy M×g progeny plant is grown from the viable, odd ploidy M×g seed of claim 12.

14. A method for producing a viable M×g seed having an odd ploidy number, the method comprising:
(a) providing a first M×g plant with an even ploidy number and a second M×g plant having a different even ploidy number from that of the first M×g plant;
(b) crossing the first M×g plant and the second M×g plant; and
(c) producing a viable M×g seed having an odd ploidy number from the mated first M×g plant and the second M×g plant.

15. The method of claim 14, wherein the mating of the first M×g plant and the second M×g plant produces a percentage of viable odd ploidy seed of at least 10% of total seed produced.

16. The method of claim 14, wherein the mating of the first M×g plant and the second M×g plant produces a yield of at least 8 pounds per acre of viable odd ploidy seed.

17. The method of claim 14, wherein the ploidy number difference between the first M×g plant and the second M×g plant is 2, 6, 8, or 10.

18. The method of claim 14, wherein the first M×g plant and the second M×g plant are selected for self-incompatibility and cross-compatibility.

19. A viable, odd ploidy M×g seed produced by the mating of the first M×g plant of claim 14 and the second M×g plant of claim 14.

20. An odd ploidy, seed-propagated M×g progeny plant that produces few or no viable seeds, wherein the odd ploidy M×g progeny plant is grown from the viable odd ploidy M×g seed of claim 19.

21. The odd ploidy, seed-propagated M×g progeny plant of claim 20, wherein less than 10%, or less than 5%, or less than 2.5%, or less than 1%, or less than 0.2%, or less than 0.1%, or less than 0.01%, of the seeds produced from the odd ploidy seed-propagated M×g progeny plants are viable.
